(19) United States
(12) Patent Application Publication
(10) Pub. No.: US 2005/0273039 A1
(43) Pub. Date: Dec. 8, 2005

(54) Tampon applicator providing low placement

(75) Inventors: Diana Lynne Gann, Lebanon, OH (US); Thomas Ward Osborn III, Clifton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/861,706

(22) Filed: Jun. 4, 2004

(51) Int. Cl. A61F 13/20

(52) U.S. Cl. 604/15

(57) ABSTRACT

A tampon applicator for positioning a tampon inside a vaginal cavity. The tampon applicator includes an outer member, an inner member, a tampon, and a tampon positioning member. The outer member is disposed co-axially with the inner member for insertion of the tampon into the vaginal cavity. The inner member includes a first end, a second end, and an interior surface. The inner member is slidable within the outer member. The tampon is at least partially housed within the inner member. The tampon positioning member is positioned within the inner member. The tampon positioning member includes a tampon pushing surface positioned between the first end and the second end of the inner member.

### FIELD OF INVENTION

This invention relates to a tampon applicator, particularly, to a tampon applicator providing low placement of a tampon within the vaginal cavity.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators generally comprise, in a simple form, a pair of coaxial hollow cylinders or tubes. The larger outer tube serves as a vaginal insertion tube and houses a tampon. The smaller inner tube serves as a plunger which ejects the tampon from the outer tube.

It has been recognized that tampon applicators of the type described above have certain deficiencies which can be readily apparent to many users of such devices. In general, the position of the tampon within the vaginal cavity can directly impact the tampon's efficiency of reducing and/or eliminating leakage. For example, conventional “push” type applicators can place a tampon too high in the vaginal cavity. The higher the tampon is placed, the greater the chance of leakage due to the tampon being above the major path of fluid flow. In other words, menstrual fluids can pass by the tampon without being absorbed.

In addition, if the position of the tampon is too high in the vaginal cavity, the tampon can extend into portions of the vaginal cavity, where, for reasons of the size and mechanical properties of the tampon, the tampon can be negatively affected by the vaginal and surrounding organs, further reducing the tampon coverage of menses flow in the vaginal cavity. Moreover, current tampon applicators are designed to “push” the tampon out of the outer tube substantially higher than the tip of the outer tube. This often causes the tampon to be deflected by the cervix/anterior fomix, resulting in an off-centered position of the tampon, and, thus, result in possible leakage from the vaginal cavity.

Alternatively, tampon applicators can place a tampon too low in the vaginal cavity. When the tampon is placed too low in the vaginal cavity, the too low position of the tampon can cause bodily discomfort to the wearer because of the pressure exerted from the sphincter muscles against the tampon. Also, if the tampon is placed too low, accidental expulsion of the tampon is a highly undesired risk.

To solve the above problem of positioning the tampon, it would be beneficial to provide a tampon applicator which properly places the tampon at a desired position in the vaginal cavity.

### SUMMARY OF THE INVENTION

The present invention encompasses a tampon applicator for positioning a tampon inside a vaginal cavity. The applicator includes an outer member, an inner member, a tampon, and a tampon positioning member. The outer member is disposed co-axially with an inner member for insertion of the tampon into the vaginal cavity. More specifically, the inner member can be slidable within the outer member. The inner member comprises a first end, a second end, and an interior surface. The tampon positioning member is positioned within the inner member. The tampon positioning member comprises a tampon pushing surface positioned between the first end and the second end of the inner member. In one embodiment, the tampon positioning member can be joined to the inner member.

The tampon is at least partially housed within the inner member. In addition, the tampon can be at least partially housed in the outer member when the tampon positioning member is at its most proximal position. For example, at least 15% of a length of the tampon remains within the inner member when the tampon positioning member is at its most proximal position. In another example, at least 10 millimeters of a length of the tampon remains within the inner member when the tampon positioning member is at its most proximal position. In another aspect, the tampon remains in contact with the tampon positioning member when the tampon positioning member is at its most proximal position.

In another embodiment, the outer member comprises an insertion end wherein the first end of the inner member is at a more proximal position than the insertion end of the outer member when the tampon positioning member is in its most proximal position. In yet another embodiment, the outer member conforms to a profile of the tampon.

All documents cited are, in relevant part, incorporated herein by reference; the citation of any document is not to be construed as an admission that it is prior art with respect to the present invention.

### BRIEF DESCRIPTION OF THE DRAWINGS

FIG. 1a is a cross-sectional view along a longitudinal axis of a tampon applicator of the present invention.

FIG. 1b is an exploded view of the tampon applicator along the longitudinal axis illustrative of the present invention before the assembly of the tampon applicator.

FIG. 2 is a cross-sectional view along a longitudinal axis of a tampon applicator partially housing a tampon inside a vaginal cavity of a wearer.

### DETAILED DESCRIPTION OF THE INVENTION

Section A will provide terms which will assist the reader in best understanding the features of the invention but not to introduce limitations in the terms inconsistent with the context in which they are used in this specification. These definitions are not intended to be limiting. Section B will discuss the tampon applicator of the present invention. Section C will discuss the tampon applicator materials.

A. Terms

As used herein, the term “tampon” refers to any type of absorbent structure that can be inserted into the vaginal cavity or other body cavities for the absorption of fluid therefrom or for the delivery of active materials, such as medicaments or moisture. A tampon can be straight or non-linear in shape, such as curved along the longitudinal axis.

Generally, there are two types of tampons. The first type of tampon is a self-sustaining tampon. Tampons are generally “self-sustaining” in that they will tend to retain their general shape and size before use. A typical self-sustaining tampon is 35-60 millimeters long, the length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 millimeters wide corresponding to the largest cylindrical cross section. The width can vary along the length of the self-sustaining tampon.

The second type of tampon is an easily “deformable, fluid-permeable bag tampon”. The deformable, fluid-permeable bag tampon consists of pieces such as absorbent chips, spheres, or fibers such that the fluid permeable bag tampon is readily deformable with a force of less than about 3 psi.

As used herein, the terms “vaginal cavity” and “within the vagina” refer to the internal genitalia of the human female in the pudendal region of the body.

As used herein, the term “hymen ring” refers to the demarcation between the vaginal cavity and external genitalia and is identified by the position of the hymen or residual tissue of the hymen.

As used herein, the term “low placement” refers to a position of the tampon inside the vaginal cavity, wherein the base of the tampon is positioned at least about 5 millimeters above the hymen ring and below the cervix. The base of the tampon does not include any overwrap, secondary absorbent member, or withdrawal cord which extends beyond the tampon's main absorbent material.

The terms “expelled” and “expulsion,” as used herein, are meant the position of the tampon after the exposed portion of the tampon is fully or partially forced out of the tampon applicator.

The term “exposed portion of the tampon,” as used herein, is meant the portion of the tampon that is revealed from the tampon applicator as measured from the most proximal end of the tampon along the longitudinal axis to the most proximal end of the insertion end of the outer member along the longitudinal axis when the tampon applicator is fully engaged.

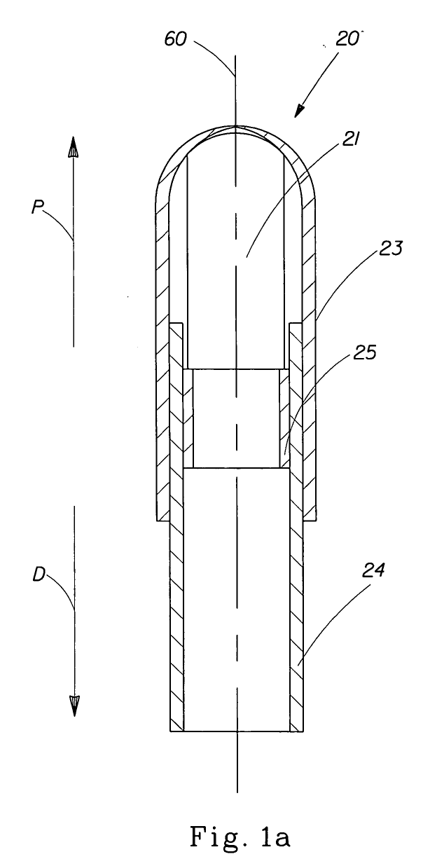

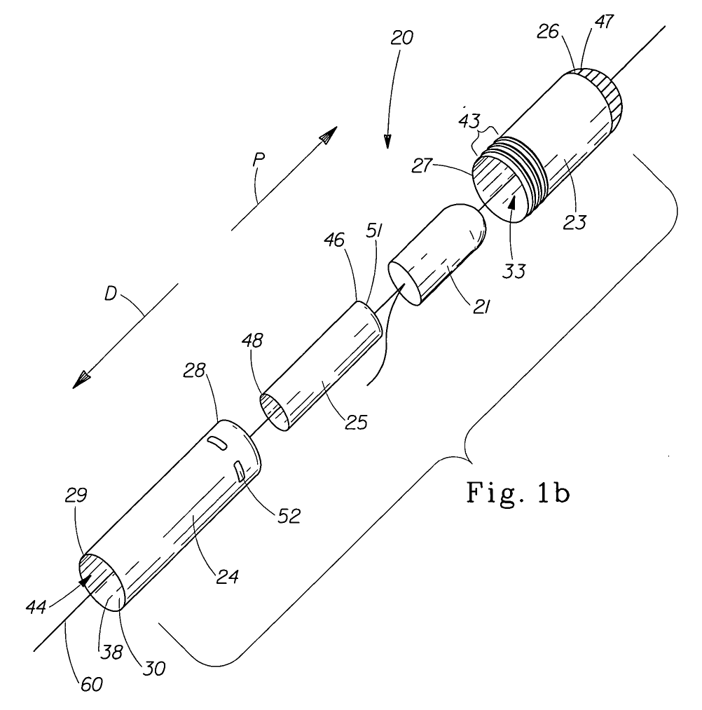

The terms “distal” and “proximal,” respectively designated P and D in FIGS. 1a and 1b, herein respectively refer to directions away from and towards the body of the tampon wearer unless otherwise specified.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Tampon Applicator of the Present Invention

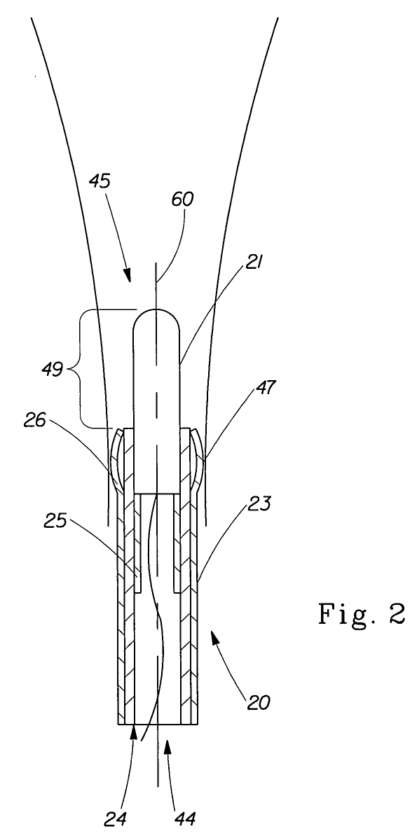

Referring to FIG. 1a, a tampon applicator 20 is shown before expulsion of an exposed portion 49 (FIG. 2) of tampon 21 along a longitudinal axis 60. Generally, the tampon applicator 20 includes an outer member 23, an inner member 24, and a tampon positioning member 25. Also, FIG. 1a shows “proximal” and “distal” designated as P and D, respectively. FIG. 1b, is an exploded view of FIG. 1a of the tampon applicator 20 along the longitudinal axis 60. The tampon applicator 20 is designed to position tampon 21 to achieve low placement in the vaginal cavity of a wearer. The tampon applicator 20 can be used with any type of tampon 21. For example, the tampon 21 could be a self-sustaining tampon or a deformable, fluid-permeable bag tampon.

Referring to FIG. 1b, the outer member 23 can be used to insert the tampon 21 into the vaginal cavity. Also, FIG. 1b shows “proximal” and “distal” designated as P and D, respectively. The outer member 23 comprises a hollow interior 33, an insertion end 26, and a second end 27 opposed to the insertion end 26. The insertion end 26 is the most proximal end to the body of a wearer of the outer member 23 along the longitudinal axis 60. The second end 27 is the most distal end of the outer member 23 along the longitudinal axis 60. The portion of the outer member 23 adjacent to the insertion end 26 can include an openable end, such as petals 47. The petals 47 are capable of opening to let the exposed portion 49 (FIG. 2) of the tampon 21 through.

The inner member 24 is used to eject the tampon 21 from the outer member 23. The inner member 24 is dimensioned to slidably move within the outer member 23, with minimal clearance therebetween. The inner member 24 has a hollow interior 30 and an interior surface 38. Alternatively, the inner member 24 can be solid or partially solid. The inner member 24 has a first end 28 and a second end 29 opposed to the first end 28. The first end 28 is the most proximal end of the inner member 24 along the longitudinal axis 60. The second end 29 is the portion of the inner member 24 in which an axial force 44 along the longitudinal axis 60 is applied to expel the exposed portion 49 (FIG. 2) of the tampon 21 through the outer member 23. The inner member 24 can have nibs 52.

The tampon positioning member 25 properly positions the tampon 21 such that during expulsion of the exposed portion 49 (FIG. 2) of the tampon 21, the tampon 21 will travel a pre-determined distance. Specifically, during expulsion of the exposed portion 49 (FIG. 2) of the tampon 21, the tampon 21 abuts the tampon pushing surface 51 of the tampon positioning member 25. The tampon positioning member 25 has a first end 46 and a second end 48 opposed to the first end 46. The first end 46 is the most proximal end of the tampon positioning member 25 along the longitudinal axis 60. The second end 48 is the most distal end of the tampon positioning member 25 along the longitudinal axis 60. The tampon positioning member 25 is positioned within the inner member 24 (see FIG. 1a) between the first end 28 and the second end 29 of the inner member 24.

To use the tampon applicator 20 of the present invention, the user will typically hold the grip region 43 located adjacent to the second end 27 of the outer member 23 with the wearer's thumb and index finger. While holding the grip region 43, the user can insert the outer member 23 of the tampon applicator 20 into the vaginal cavity and apply an axial force 44 sufficient enough to push the inner member 24 into the outer member 23.

Referring to FIG. 2, when the exposed portion 49 of the tampon 21 begins to be pushed out of the insertion end 26 of the outer member 23 with the axial force 44 applied along the longitudinal axis 60, petals 47, if included, begin to open. When the tampon positioning member 25 travels a pre-determined distance, the exposed portion 49 of the tampon 21 is revealed out of the tampon applicator 20. Because the exposed portion 49 of the tampon 21 is revealed in the vaginal cavity 45, during removal of the tampon applicator 20 from the vaginal cavity, the body tissues of a wearer continue to hold onto the exposed portion 49 of tampon 21. Because the force of the body tissues which hold onto the exposed portion 49 of tampon 21 is greater than the inner member's 24 force used to house the tampon 21, the tampon applicator 20 exits the vaginal cavity without the tampon 21, i.e., leaving the tampon 21 behind. This results in low placement of the tampon 21 within the vaginal cavity.

In addition to delivering menstrual tampons into the vaginal cavity, it should be noted that the tampon applicator of the present invention can be used to deliver any other type of absorbent or nonabsorbent object to any suitable cavity. For example, the tampon applicator of the present invention could be used to insert incontinence inserts. An “incontinence insert,” as used herein, refers to devices specifically designed, configured, and/or adapted for placement into a vagina in order to reduce the occurrence and/or severity of female urinary incontinence. While incontinence inserts are typically made of non-absorbent materials, at least partially absorbent materials may also be used. However, because there is no intent to absorb bodily fluids, and because the incontinence inserts are adapted and configured to provide structural support to the musculature and body tissues located near the urethra-vaginal myfascial area, incontinence inserts are readily distinguishable from tampons.

Below will provide more detail of each component of the tampon applicator of the present invention.

i. Outer Member

Referring to FIG. 1b, typically, the outer member 23 can be used to handle or grip the tampon applicator 20 during the insertion into the vaginal cavity. The outer member 23 is external to the inner member 24. The outer member 23 has a hollow interior 33, an insertion end 26, and a second end 27 opposed to the insertion end 26. The insertion end 26 is the most proximal end of the outer member 23. In addition, the insertion end 26 is a portion of the outer member 23 in which the tampon applicator 20 is first inserted into the vaginal cavity and it is the end from which the exposed portion 49 (FIG. 2) of the tampon 21 is expelled. The second end 27 is the most distal portion of the outer member 23 along the longitudinal axis 60. Grip region 43 is the portion of the outer member 23 in which the user can handle or grip the tampon applicator 20. At least a portion of the hollow interior 33 of the outer member 23 can engage with at least a portion of the inner member 24.

The manufacturer of the tampon applicator 20 can vary the size of the outer member 23. The size of the outer member 23 can be determined primarily by the dimensions of the tampon 21. Specifically, the diameter of the outer member 23 can be varied to accommodate different absorbency tampons. Generally, the outer member 23 can include an inner diameter of from about 6 millimeters to about 20 millimeters and a wall thickness of from about 0.4 millimeter to about 2.0 millimeters. Generally, the inner diameter of the outer member 23 should be suitably greater than the diameter of the tampon 21 to prevent the outer member 23 from interfering with the removal of the tampon 21 from the outer member 23. Moreover, the inner diameter of the outer member 23 can have varying diameters and shapes to conform to the profiled shape of the enclosed tampon 21.

Also, the manufacturer of the tampon applicator 20 can vary the length of the outer member 23. Generally, the outer member 23 should be of a sufficient length to house at least a portion of the tampon 21 prior to the expulsion of the tampon 21 from the tampon applicator 20 into the vaginal cavity. More specific examples are described below.

In addition, the manufacturer of the tampon applicator 20 can vary the shape of the outer member 23. It is further noted herein that the shape of the outer member 23 can vary as long as a portion of the inner member 24 can be slideable within the outer member 23. Moreover, the outer member 23 is in no way limited by the shape that it can assume except that the shape should not hinder directional expulsion of the tampon 21. One skilled in the art can imagine that the outer member 23 might be cylindrical or curved like a banana or any other suitable shape as long as any such shape would work effectively to allow the exposed portion 49 (FIG. 2) of tampon 21 to expel from the tampon applicator 20, and comfortably insert the tampon 21 into the vaginal cavity. The outer member 23 can be of any suitable cross-sectional shape. For example, suitable cross-sectional shapes can include, but are not limited to, circular, oval, flattened circular, elliptical, and any combination thereof.

In addition, the second end 27 can also have many designs. For example, the second end 27 can be scalloped at the second end 27 and/or have a feathered thickness edge.

Furthermore, the outer member 23 can contain the grip region 43 adjacent to the second end 27 as noted above. The grip region 43 can provide for secure handling of the outer member 23. The perimeter of the grip region 43 can take essentially any desired shape, including oval, circular, and various other geometric forms. The gripping region 43 can be defined by outward projections or raised surfaces, created by impressing or compressing the surfaces. Referring again to FIG. 1b, the grip region 43 is substantially circularly shaped, but it can also take on more angular formations such as squared. The surface of the grip region 43 can be any kind of surface known in the art. This surface can provide a desired frictional resistance for the fingers during the insertion of the tampon applicator 20 into the body. Moreover, this surface area can have, for example, a dove pattern created via surface texturing, impressions, and/or indentations.

Furthermore, it is preferable that the grip region 43 be of sufficient dimension to substantially provide a comfortable grip for the user. As used in the specification, the term “user's grip” means any way of holding the tampon applicator 20 in a hand, e.g., between a thumb and a finger. The grip region 43 need not necessarily extend completely around the perimeter of the outer member 23. For example, a space can be provided for a decorative marking or a Trademark character.

ii. Inner Member

In FIG. 1b, inner member 24 can be designed to store at least a portion of a tampon 21 within its hollow interior 30 either during the expulsion and/or before the expulsion of the exposed portion 49 (FIG. 2) of the tampon 21 from the insertion end 26 of the outer member 23. The inner member 24 has a hollow interior 30, a first end 28, and a second end 29 opposed to the first end 28. The first end 28 is the most proximal end of the inner member 24 along the longitudinal axis 60. The second end 29 is the most distal end of the tampon applicator 20 along the longitudinal axis 60. Moreover, the second end 29 is the portion of the inner member 24 in which the axial force 44 is applied to expel the exposed portion 49 (FIG. 2) of the tampon 21 from the outer member 23.

The tampon positioning member 25 is positioned within the inner member 24. Preferably, the tampon positioning member 25 is joined to the inner member 24. The tampon positioning member 25 can be joined to the inner member 24 in any way known in the art. Specifically, the tampon positioning member 25 can be joined to the interior surface 38 of the inner member 24.

The manufacturer of the tampon applicator 20 can vary the shape of the inner member 24 as long as any such shape would work effectively to partially contain tampon 21. Notably, the inner member 24 is shaped such that at least a portion of the tampon 21 is housed in or is partially contained by the inner member 24 before the expulsion or during the expulsion of the exposed portion 49 (FIG. 2) of the tampon 21 from the tampon applicator 20. One skilled in the art can imagine other shapes of the inner member 24, for example, cylindrical or curved like a banana or any other suitable shape possible. The inner member 24 can be of any suitable cross-sectional shape including, but are not limited to, circular, oval, flattened circular, elliptical, and any combinations thereof.

The size of the inner member 24 can be determined primarily by the dimensions of tampon 21. Specifically, the diameter of the inner member 24 can be varied to accommodate different absorbency tampons. For example, higher absorbency tampons can have larger diameters resulting in the inner diameter of the inner member 24 having a larger diameter to house the higher absorbent tampon. In addition, the inner diameter of the inner member 24 can have varying diameters and shapes to conform to the profiled shape of the enclosed tampon 21.

The length of the inner member 24 can also vary. Generally, the inner member 24 should be of a sufficient length to house a substantial portion of the tampon 21 within the hollow interior 30 of the inner member 24 properly aiding in the expulsion of the exposed portion 49 (FIG. 2) of tampon 21 from the tampon applicator 20. For example, the length of the inner member 24 can be from about 43 millimeters to about 90 millimeters.

Optionally, it can be seen that inner member 24 can have nibs 52 provided on the perimeter thereof located adjacent to the first end 28 of inner member 24. If nibs 52 are included, the inner member 24 should fit within the outer member 23. The nibs 52 can be adapted to engage with the grooves on the inside surface (not shown) of outer member 23 thereby providing a number of possible predetermined relative positions for the outer member 23 and for the inner member 24. Nibs 52 prevent the inner member 24 from too easily being pushed into the outer member 23 (resulting in premature expulsion of the tampon 21) should the tampon applicator 20 be mishandled before the use thereof.

iii. Tampon Positioning Member

The tampon positioning member 25 positions the tampon 21 along the length of the inner member 24 so that during expulsion of the exposed portion 49 (FIG. 2) of the tampon 21, the tampon 21 will travel a pre-determined distance. This predetermined distance ultimately results in low placement of the tampon 21 in the vaginal cavity. Moreover, the predetermined distance that the tampon 21 travels occurs when the tampon positioning member 25 is at its most proximal position.

Referring to FIG. 1b, the tampon positioning member 25 has a first end 46 and a second end 48 opposed to the first end 46. The first end 46 is the most proximal end of the tampon positioning member 25 along the longitudinal axis 60. The second end 48 is the most distal end of the tampon positioning member 25 along the longitudinal axis 60. Also, the first end 46 includes the tampon pushing surface 51. The tampon pushing surface 51 is any part of the tampon positioning member 25 which abuts the tampon 21 during expulsion of the exposed portion 49 (FIG. 2) of tampon 21. In one non-limiting example, the tampon pushing surface 51 abuts the sides of tampon 21 during expulsion of the exposed portion 49 (FIG. 2) of tampon 21. In this non-limiting example, the tampon pushing surface 51 provides the necessary force along the sides of tampon 21 to expel the exposed portion 49 (FIG. 2) of tampon 21. The tampon pushing surface 51 can be any shape as long as such shape would work effectively to provide enough force to expel the exposed portion 49 (FIG. 2) of the tampon 21. The first end 46 of the tampon positioning member 25 is the tampon pushing surface 51 for this embodiment. In one embodiment, the tampon pushing surface 51 is flat. In another embodiment, the tampon pushing surface 51 has an uneven surface.

The tampon positioning member 25 is placed within inner member 24. In one embodiment, the tampon positioning member 25 can be located between the first end 28 and the second end 29 of the inner member 24. The tampon positioning member 25 can be joined to the inner member 24 in any way known in the art. The tampon positioning member 25 can also be monolithic with the inner member 24, for example, by molding the tampon positioning member 25 and the inner member 24 as one piece. In one non-limiting example, the tampon positioning member 25 can be joined to the inner member 24 by friction fitting that snaps parts together, gluing, and/or melting. In another non-limiting example, the tampon positioning member 25 can also be a continuation of the inner member 24 by folding or compressing a portion of the inner member 24 and turning the inner member 24 inwards upon itself to create a tube inside a tube. In yet another non-limiting example, the tampon positioning member 25 can also be a string, wire, or a cord which has a first end which is connected to one side of the inner member 24 and a second end connected to a second side of the inner member 24. In yet another non-limiting example, the tampon positioning member 25 can be a string, wire, or a cord which has only a first end which is connected to one side of the inner member 24 and a second end which is adjacent to the tampon 21 during expulsion of the tampon 21.

The tampon pushing surface 51 positions tampon 21 to expel the exposed portion 49 of the tampon 21 when force is applied to the tampon pushing surface 51 by the user. More specifically, the tampon pushing surface 51 can be located from about 0 millimeters to about 45 millimeters from the insertion end 26 of the inner member 24 along the longitudinal axis 60.

Referring to FIG. 2, during the expulsion, at least a portion of the tampon 21 is adjacent to the tampon positioning member 25. In other words, when the tampon positioning member 25 is at its most proximal position, the tampon positioning member 25 is in contact with the tampon 21. Specifically, the tampon pushing surface 51 applies the force required to expel the exposed portion 49 (FIG. 2) of the tampon 21. However, the tampon 21 may or may not be in contact with the tampon pushing surface 51 before the expulsion of the exposed portion 49 of the tampon 21 from the tampon applicator 20.

It is further noted herein that the shape of the tampon positioning member 25 can vary as long as the tampon positioning member 25 aids in positioning the tampon 21 along the length of the outer member 23 to provide low placement of the tampon 21 within the vaginal cavity. In one embodiment, the tampon positioning member includes an elongated tampon positioning member of a generally tubular shape. Alternatively, the tampon positioning member can be a rod. In another embodiment, the tampon positioning member can be a tube. In yet another embodiment, the tampon positioning member 25 can be slideable within the outer member 23. In another alternative embodiment, the tampon positioning member can also comprise projections extending inward from the hollow interior 30 of the inner member 24. The projections can be of any shape or size as long as the projections allow the tampon 21 to be in contact with the projections during the expulsion of the exposed portion 49 (FIG. 2). The projections can be fixedly joined to the inner member 24 in any way known in the art.

It is further noted herein that the length of the tampon positioning member 25 can vary as long as the tampon pushing surface 51 aids the tampon 21 to be located in a position within the inner member 24 that will provide low placement within the vaginal cavity. In one non-limiting example, the length of the tampon positioning member 25 can be from about 39 millimeters to about 60 millimeters.

C. Tampon Applicator Materials

Different tampon applicator parts can be constructed from different materials and processes. The tampon applicator or any part of the tampon applicator can be formed of a spirally wound, convolutedly wound, or longitudinally seamed hollow tube that is formed from paper, paperboard, cardboard, or any combinations thereof. The tampon applicator or any part of the tampon applicator can also be injection molded, extruded, or formed from flexible plastic, such as thermoformed from plastic sheet or folded or wound from plastic film.

The tampon applicator or any part of the tampon applicator can be constructed from a single ply of material or be formed from two or more plies that are bonded together to form a laminate. The use of two or more plies or layers is preferred for it enables the manufacturer to use certain materials in the various layers that can enhance the performance of the tampon applicator or any part of the tampon applicator. When two or more plies are utilized, all the plies can be spirally wound, convolutedly wound, or longitudinally seamed to form an elongated cylinder. The tampon applicator or any part of the tampon applicator can be constructed using a smooth thin ply of material on the outside or exterior surface that surrounds a coarser and possibly thicker ply. When the tampon applicator or any part of the tampon applicator contains at least three plies, the middle ply can be the thicker ply, and the interior and exterior plies can be smooth and/or slippery to facilitate the expulsion of the tampon and to facilitate the insertion of the tampon applicator or any part of the tampon applicator into a woman's vagina. By sandwiching a thick, coarser ply of material between two thin, smooth plies, a tampon applicator or any part of the tampon applicator can be provided which is very functional. The tampon applicator or any part of the tampon applicator should contain one to four plies, although more plies can be utilized if desired.

The plies forming the tampon applicator or any part of the tampon applicator can be held together by an adhesive, such as glue, heat, pressure, ultrasonic, or any combinations thereof. The adhesive can be either water-soluble or water-insoluble. A water-soluble adhesive is preferred for environmental reasons in that the tampon applicator or any part of the tampon applicator will quickly break apart when it is immersed in water. Such immersion will occur should the tampon applicator or any part of the tampon applicator be disposed of by flushing it down a toilet. Exposure of the tampon applicator or any part of the tampon applicator to a municipal's waste treatment plant wherein soaking in water, interaction with chemicals, and agitation all occur, will cause the tampon applicator or any part of the tampon applicator to break apart and evenly disperse in a relatively short period of time.

### EXAMPLES

The following is a listing of examples illustrating various embodiments of the present invention. It would be obvious to those skilled in the art that various other changes and modifications can be made without departing from the spirit and scope of the invention.

### Example 1

The tampon applicator of the present invention can comprise the outer member of the length of 70 millimeters, the inner member of the length of 73 millimeters, a tampon positioning member of the length of 56 millimeters, and a tampon of the length of 44 millimeters. The tampon pushing surface is located 17 millimeters from the first end of the inner member along the longitudinal axis.

### Example 2

The tampon applicator of the present invention can comprise the outer member of the length of 67 millimeters, the inner member of the length of 68 millimeters, a tampon positioning member of the length of 57 millimeters, and a tampon of the length of 38 millimeters. The tampon pushing surface is located 11 millimeters from the first end of the inner member along the longitudinal axis.

### Example 3

The tampon applicator of the present invention can comprise the outer member of the length of 70 millimeters, the inner member of the length of 73 millimeters, a tampon positioning member of the length of 45 millimeters, and a tampon of the length of 46 millimeters. The tampon pushing surface is located 28 millimeters from the first end of the inner member along the longitudinal axis.

### Example 4

The tampon applicator of the present invention can comprise the outer member of the length of 58 millimeters, the inner member of the length of 62 millimeters, a tampon positioning member of the length of 48 millimeters, and a tampon of the length of 30 millimeters. The tampon pushing surface is located 14 millimeters from the first end of the inner member along the longitudinal axis.

### Example 5

The tampon applicator of the present invention can comprise the outer member of the length of 66 millimeters, the inner member of the length of 68 millimeters, a tampon positioning member of the length of 51 millimeters, and a tampon of the length of 45 millimeters. The tampon pushing surface is located 17 millimeters from the first end of the inner member along the longitudinal axis.

### Example 6

The tampon applicator of the present invention can comprise the outer member of the length of 55 millimeters, the inner member of the length of 55 millimeters, a tampon positioning member of the length of 45 millimeters, and a tampon of the length of 30 millimeters. The tampon pushing surface is located 10 millimeters from the first end of the inner member along the longitudinal axis.

### Example 7

The tampon applicator of the present invention can comprise the outer member of the length of 65 millimeters, the inner member of the length of 60 millimeters, a tampon positioning member of the length of 2 millimeters, and a tampon of the length of 45 millimeters. The tampon pushing surface is located 15 millimeters from the first end of the inner member along the longitudinal axis.

### Example 8

The tampon applicator of the present invention can comprise the outer member of the length of 55 millimeters, the inner member of the length of 58 millimeters, a tampon positioning member of the length of 38 millimeters, and a tampon of the length of 44 millimeters. The tampon pushing surface is located 20 millimeters from the first end of the inner member along the longitudinal axis.

### Example 9

The tampon applicator of the present invention can comprise the outer member of the length of 66 millimeters, the inner member of the length of 68 millimeters, a tampon positioning member of the length of 2 millimeters, and a tampon of the length of 45 millimeters. The tampon pushing surface is located 17 millimeters from the first end of the inner member along the longitudinal axis.

### Example 10

The tampon applicator of the present invention can comprise the outer member of the length of 68 millimeters, the inner member of the length of 60 millimeters, a tampon positioning member of the length of 45 millimeters, and a tampon of the length of 45 millimeters. The tampon pushing surface is located 0 millimeters from the first end of the inner member along the longitudinal axis.

All documents cited in the Detailed Description of the Invention are incorporated, in relevant part, herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A tampon applicator for positioning a tampon inside a vaginal cavity, said applicator comprising:
a) an outer member disposed co-axially with an inner member for insertion of said tampon into said vaginal cavity, said inner member comprising a first end, a second end, and an interior surface;
b) said tampon at least partially housed within said inner member;
c) said inner member being slidable within said outer member;
d) a tampon positioning member being positioned within said inner member, said tampon positioning member comprising a tampon pushing surface positioned between said first end and said second end of said inner member.

2. The tampon applicator of claim 1 wherein said tampon positioning member is joined to said inner member.

3. The tampon applicator of claim 1 wherein said tampon is at least partially housed in said outer member when the tampon positioning member is at its most proximal position.

4. The tampon applicator of claim 1 wherein said tampon is at least partially housed within said inner member when the tampon positioning member is at its most proximal position.

5. The tampon applicator of claim 4 wherein at least 15% of a length of said tampon remains within said inner member when the tampon positioning member is at its most proximal position.

6. The tampon applicator of claim 4 wherein at least 10 millimeters of a length of said tampon remains within said inner member when the tampon positioning member is at its most proximal position.

7. The tampon applicator of claim 1 wherein said outer member comprises an insertion end, wherein said first end of said inner member is at a more proximal position than said insertion end of said outer member when said tampon positioning member is in its most proximal position.

8. The tampon applicator of claim 1 wherein said tampon remains in contact with said tampon positioning member when said tampon positioning member is at its most proximal position.

9. The tampon applicator of claim 1 wherein said outer member conforms to a profile of said tampon.
